(19) United States
(12) Patent Application Publication
(10) Pub. No.: US 2013/0053862 A1
(43) Pub. Date: Feb. 28, 2013

(54) DEVICE FOR PRODUCING AN ANASTOMOSIS CONNECTION

(71) Applicant: Cornelis Antonius Franciscus Tulleken, Utrecht (NL)

(72) Inventor: Cornelis Antonius Franciscus Tulleken, Utrecht (NL)

(73) Assignee: AMJ bv, Utrecht (NL)

(21) Appl. No.: 13/496,328

(22) PCT Filed: Nov. 19, 2010
(86) PCT No.: PCT/NL2010/050778

§ 371 Date: Jul. 10, 2012

(30) Foreign Application Priority Data
Nov. 19, 2009 (NL) PCT/NL2009/000228

(51) Int. Cl. A61B 17/11; A61F 2/06

(52) U.S. Cl. 606/108; 606/153

(57) ABSTRACT

The invention relates to a device for producing an ETS anastomosis connection and/or a STS anastomosis connection, comprising a ring (10) with two pins (11). Each pin has a free end (12) and a fixed end (13) which is secured to the ring; the pins extending, viewed from the fixed end toward the free end, next to and set apart from each other in the same direction. The pins each have a clamping section where the pin extends, in a first position, parallel to and along said ring from a first side of the ring to a second side of the ring that is situated diametrically opposite said first side in order to be able to clamp tissue of the wall of the receiving vessel between the ring and the clamping sections. The clamping sections of the pins are, in a second position, at an angle with respect to the plane defined by the ring in order to form, together with the ring, a jaw which widens, viewed in the direction from the fixed ends toward the free ends. The pins are swivellable, with respect to the ring, by means of a swivelling movement from the second position to the first position for closing the jaw.

The present invention relates to a device for producing an ETS anastomosis connection (ETS=end to side) and/or a STS anastomosis connection (STS=side to side) between a graft vessel and the side wall of a receiving vessel (also referred to as the target vessel), comprising a ring with two cantilever pins, each pin having a free end and a fixed end which is secured to the ring; the pins extending, viewed from the fixed end toward the free end, next to and set apart from each other in the same direction; the pins each having a clamping section where the pin extends, in a first position, parallel to and along said ring from a first side of the ring to a second side of the ring that is situated diametrically opposite said first side in order to be able to clamp tissue of the wall of the receiving vessel between the ring and the clamping sections. A device of this type is known from WO 2009/123434.

Although it is not a securing device, a ring without pins is known from EP 750,467. Said publication describes what is referred to as the ELANA technique (Elana=Excimer Laser Assisted Nonocclusive Anastomosis). The present invention is particularly intended for use with the ELANA technique, but can also be used for other medical techniques.

The ELANA technique is used to produce an anastomosis without temporarily closing off the receiving vessel, also referred to as the target vessel. With the ELANA technique, the through-flow of the receiving vessel is not interrupted while the anastomosis is performed. Neurosurgeons, as well as other surgeons, use this technique to perform bypass operations. However, the ELANA technique can also be applied in other areas of surgery, such as cardiac surgery and general vascular surgery. The present invention can also be used in the case of these medical procedures.

The ELANA technique involves placing an implantable ring around the distal end of a graft vessel, folding the section of the wall of the graft vessel, which is distal from the ring, back over approximately 90° or approximately 180° around the ring in order to form a distal mouth of the graft vessel reinforced by the ring. Said reinforced distal mouth is then attached to the wall of the receiving vessel, usually by suturing the graft vessel to the receiving vessel by means of approximately 8 stitches. Depending on the ability of the surgeon, the location of the receiving vessel and the condition of the patient, said attachment generally takes between 15 minutes and one hour or more. During this attachment procedure, there is still no through-flow connection between the graft vessel and the receiving vessel. After the attachment procedure, a laser catheter is inserted into the graft vessel in order to create a through-flow connection between the graft vessel and the receiving vessel by burning away the wall part of the receiving vessel which is situated in front of the distal mouth of the graft vessel. Blood flows through the receiving vessel during the entire operation, as a result of which temporary closure of the receiving vessel is prevented.

The securing device according to the preamble of claim 1 is known from WO 2009/123434. The embodiments depicted therein comprise a ring which is to be imagined divided into two halves and which is provided with two pins. The pins are secured at one end, referred to as the fixed end, to the ring without the pins being in this case movable with respect to the ring. From the fixed end, the pins extend next to each other, in the same direction, to the free ends of the pins. Just as in the present invention, the pins are cantilever pins; that is to say, the pins extend from the fixed end to the free end unimpeded—i.e. without being supported between the fixed end and the free end or at the free end—and are sufficiently rigid so as not to sag, or to sag only slightly, under their own weight as they extend horizontally. As described in WO 2009/123434, the pointed ends of the pins are inserted, during use, through the wall of the receiving vessel via entrance holes created by the pins, after which the overall entity formed by the ring and pins is slid further in the longitudinal direction of the receiving vessel until the fixed ends of the pins are located in the entrance holes or have just passed therethrough. During the further sliding of the ring with pins in the longitudinal direction of the receiving vessel, the wall of the receiving vessel slides in the meantime into the gap which is formed between the ring and the section running therealong—referred to as the clamping section—of the pins. The fixing of the graft vessel to the receiving vessel is thus greatly simplified, compared to the conventional technique of (manual) suturing. However, the inward sliding into the gap does not always proceed equally straightforwardly and the leak-tightness of the connection between the graft vessel and the receiving vessel is not always equally good.

The object of the present invention is to further improve a securing device according to the preamble of claim 1 in order to be able to implement the procedure of securing the graft vessel to the receiving vessel rapidly and more simply, and to establish in the process a reliable connection between the graft vessel and receiving vessel.

According to the invention, this object is achieved by providing a device for producing an ETS anastomosis connection between an end of a graft vessel and the side wall of a receiving vessel and/or a STS anastomosis connection between a graft vessel and the side wall of a receiving vessel, comprising a ring with two pins; each pin having a free end and a fixed end which is secured to the ring; the pins extending, viewed from the fixed end toward the free end, next to and set apart from each other in the same direction; the pins each having a clamping section where the pin extends, in a first position, parallel to and along said ring from a first side of the ring to a second side of the ring that is situated diametrically opposite said first side in order to be able to clamp tissue of the wall of the receiving vessel between the ring and the clamping sections; the device according to the invention being characterized in that the clamping sections of the pins are, in a second position, at an angle with respect to the plane defined by the ring in order to form, together with the ring, a jaw which widens, viewed in the direction from the fixed ends toward the free ends; and in that the pins are swivellable, with respect to the ring, by means of a swivelling movement from the second position to the first position for closing the jaw. As stated, the pins are in accordance with the invention cantilevered just as in WO 2009/123434; however, in WO 2009/123434, the pins are secured, directly or indirectly, to the ring so as to be rigid with respect to the ring, whereas the pins in accordance with the invention are secured, directly or indirectly, to the ring in a manner allowing swivelling of the pins with respect to the ring.

In the device according to the invention, the securing to the receiving vessel can take place with the pins in the second position. The pins are, in the second position, at an angle with respect to the plane defined by the ring and thus form, together with the ring, an open jaw. This open jaw widens, viewed from the fixed ends of the pins toward the free ends. Once the entrance holes are formed, the pins can thus easily be slid further inward because the wall of the receiving vessel can now easily be accommodated in the open jaw which is in the second position. Once the pins have been slid sufficiently far inward, the pins can be swivelled from the second position (‘open jaw’) to the first position (‘closed jaw’) in order to enclose the tissue situated between the pins and ring (i.e. the wall of the receiving vessel that is present between the ring and the pins and optionally the wall of the graft vessel that is present between the ring and the pins), in particular to clamp it in between, on the one hand, the clamping sections of the pins and, on the other hand, the ring. Although other ways will also be discussed hereinafter, the pins can optionally be swivelled by hand—by squeezing-in the receiving vessel—from the second position to the first position and be fixed, or securely clamped, in the first position by means of, for example, stitching wire (for example: the wire is inserted into the receiving vessel, pulled through under the pin or pins, fed back out of the vessel in order then to be tightened and tied in a knot). A suturing operation of this type requires little time compared to the conventional suturing of an ETS anastomosis or a STS anastomosis connection.

The accommodating of the wall of the receiving vessel in the jaw proceeds particularly straightforwardly if, in the second position, the angle of the clamping sections of the pins with respect to the plane defined by the ring is at least 5°. If the aforementioned angle is at least 10°, the surgeon can have a good view at the location of the operation. This enables the surgeon to see clearly what he is doing during the inserting of the pins into the receiving vessel and the subsequent sliding of the device according to the invention into place.

With a view to a large range of the jaw—i.e. a large overlap of the pin (and therefore the clamping section of the pin) with the ring—viewed parallel to the plane defined by the ring, the distance from the swivel axis of the aforementioned swivelling movement to a transverse axis, running parallel to the swivel axis, through the centre of the ring is at least 70%, more particularly at least 90%, of the radius of the ring. Given that each pin extends from a first side of the ring to a diametrically opposing side of the ring, this means that when the jaw is closed, in the first position, clamping-in takes place over at least 85% or at least 95% respectively of the diameter of the ring, assuming that the pins extend, viewed from the fixed end thereof, up to or past the diametrically opposing side of the ring. The larger the range of the jaw, the better, in the first position, the sealing of the graft vessel to the receiving vessel will be.

With a view to a maximum range of the jaw, viewed parallel to the plane defined by the ring, the distance from the swivel axis of the aforementioned swivelling movement to a transverse axis, running parallel to the swivel axis, through the centre of the ring is greater than the radius of the ring. The greater the aforementioned distance is, the easier it will be to implement—by leverage—a relatively high clamping force. In this connection, viewed parallel to the plane defined by the ring, the distance from the swivel axis of the aforementioned swivelling movement to a transverse axis, running parallel to the swivel axis, through the centre of the ring will be greater than 2× the radius of the ring.

In order to be able to hold the pins in a simple manner, without for example fixing the pins by means of the aforementioned stitching wire, in the first position, the device according to the invention is, according to a further embodiment, provided with a holding mechanism configured to counteract return of the pins from the first position to the second position. A holding mechanism of this type can be embodied in a broad range of ways, such as in the ways discussed hereinafter and also in other ways. A holding mechanism of this type will be pre-fitted to the device according to the invention in advance, such as prior to the operation or even already during the manufacture of the device. A holding mechanism of this type can even be formed as a single entity integrated with the ring and pins.

In order, on the one hand, to prevent accidental opening of the jaw and, on the other hand, to ensure robust clamping of the jaw, the holding mechanism can comprise a lock configured to lock the pins in the first position. A lock of this type can for example comprise a snap mechanism or a ratchet mechanism (i.e. a snap mechanism, the snapping of which is audible by way of a clicking sound) with one or more locking positions.

In order to allow automatic closing of the jaw, the holding mechanism according to a further embodiment comprises a tensioning member which pre-tensions the pins from the second position in a pre-tensioning direction directed toward the first position. In order in this case to ensure sufficient clamping in the closed position of the jaw, the first position, the tensioning member pre-tensions in this case, in accordance with a more detailed embodiment, the pins (also) in the first position in the aforementioned tensioning direction. If desired, the pre-tensioning force can be temporarily absorbed in the second position (in order to prevent the pins from swivelling prematurely from the second position to the first position) by means of an interposed standard operating instrument or by means of the surgeon's fingers.

According to a further embodiment, the tensioning member comprises a bent, resilient sheet or wire part, in particular a sheet or wire part made of metal. A sheet or wire part of this type allows a relatively high pre-tensioning force to be developed without taking up much space. Furthermore, a sheet or wire part of this type allows the entire device according to the invention to be formed from a single piece of sheet material, for example by cutting out, by means of an etching technique, laser technique or otherwise, from a flat piece of metal sheet (made for example of titanium, a titanium alloy or other metal or metal alloy suitable for implantation in the human body), a suitably shaped piece and then bending this piece into the desired shape by bending operations and (afterwards or in the meantime) optionally also subjecting it to heat treatments. According to a further embodiment, the tensioning force is in this case to be increased if the sheet or wire part comprises one or more arc of a circle-shaped windings.

With a view to a reliable and clear release of the pre-tensioning from the second position, the device according to the invention comprises in a further embodiment a release member which is operable from a blocking position to a release position, wherein the pins, in the blocking position, are in the second position and are prevented from swivelling from the second position to the first position and, in the release position, are released to swivel to the first position.

According to a further embodiment of the invention, each pin is provided, between the clamping section of the pin and the fixed end, with a first bending part which, viewed from the clamping section toward the fixed end, bends away in the direction of the plane defined by the ring, in particular over an angle of at least 20°. A bending part of this type, in particular if it bends away sharply over an angle of at least 20°, helps to ensure that the ring, when the device according to the invention is secured to the receiving vessel, is situated parallel to the longitudinal direction of the receiving vessel, thus reducing the risk of leakage during the connecting of the graft vessel and receiving vessel. It also promotes a more stable fixing to the receiving vessel as a result of the fact that there is less risk of the device according to the invention shifting once it has been fitted to the receiving vessel; this, again, helps to reduce the risk of leakage. With a view to the foregoing, it is particularly advantageous if the first bending part is located, in the first position and viewed transversely to the plane defined by the ring, substantially just below the ring (that is to say, the body which forms the ring).

In a corresponding manner as with regard to the first bending part, the same effects may be achieved, or else achieved even better in combination with the first bending part, if each pin is provided, between the clamping section of the pin and the free end, with a second bending part which, viewed from the clamping section toward the free end, bends away in the direction of the plane defined by the ring, in particular over an angle of at least 20°. In a corresponding manner as in the first bending part, it is in this case too particularly advantageous if the second bending part is located, in the first position and viewed transversely to the plane defined by the ring, substantially just below the ring (that is to say, the body which forms the ring).

A further embodiment of the invention with the aforementioned second bending part provides for the pin to continue, in particular substantially straight, from the second bending part in the direction of the plane defined by the ring, up to the free end and wherein the free end is provided with a point which, in the first position, ends in the plane defined by the ring or protrudes above the plane defined by the ring. If this point protrudes, in the first position, above the plane defined by the ring, the point can be anchored in the end of the graft vessel that is connected to the receiving vessel and thus help to fix the entity as a whole. If this point ends in the plane defined by the ring, the point will be anchored in the wall of the graft vessel and thus help to fix the entity as a whole.

Another further embodiment of the invention with the aforementioned second bending part provides for, viewed in the direction of the free end of the pin, the second bending part to be followed by a third bending part which bends the pin back, in the opposite direction, to a section running substantially parallel to the clamping section. During fitting to the receiving vessel, this section of each pin that is bent back to substantially parallel to the clamping section may be led back, via an exit hole formed in the wall of the receiving vessel, to the outer rim of the receiving vessel in order to enter into abutment along said outer rim. This helps to position the device according to the invention more stably on the receiving vessel. In order in this case to extensively prevent, after fitting, tilting of the ring with respect to the receiving vessel, provision is in this case made, in accordance with a further embodiment, for the section which is bent back to substantially parallel to the clamping section to extend, in the first position, in the plane defined by the ring. Crushing of the wall of the receiving vessel (which could lead to reactive forces on the pins and tilting of the ring at sufficient blood pressure in the receiving vessel) is thus prevented. In order in this case to reduce the risk of damage to the receiving vessel, the free end is in this case, in accordance with a further embodiment, provided with a point which protrudes, in the first position, from the section which is bent back to substantially parallel to the clamping section obliquely upward above the plane defined by the ring.

According to the invention, the anchoring of the graft vessel to the ring may be improved if the ring is provided with a plurality of protrusions which are attached distributed over the circumference, stand obliquely with respect to the axial and radial direction of the ring and point in a direction away from the pins. The ring is in this case in the form of a sort of crown and the protrusions can be anchored in the section of the graft vessel that is turned back around the ring.

According to a further embodiment, the device further comprises a graft vessel which is inserted with a distal end through the ring. The pins can in this case be inserted through the section of the wall of the graft vessel that is situated distally from the ring, but preferably the pins extend through incisions formed in the distal end of the graft vessel. The graft vessel in this embodiment can be both an artificial vessel, such as an artificial blood vessel, and a (natural) donor vessel which can be of animal origin or of human origin. Preferably, the graft vessel will derive from the patient himself or be an artificial vessel. The graft vessel and the remainder of the device according to the invention may be prepared entirely outside the patient's body to form an assembly. This can take place in a laboratory remotely from the patient, even while the patient is not being treated, for example is at home. However, this can also take place next to the patient while the patient is lying on the operating table for the operation. In case the graft vessel derives from the patient, it is usually a vessel derived from the leg of the patient, which, after it has been fully separated from the patient, is prepared for use elsewhere in the same patient, like at the heart or head of the patient.

According to a further embodiment of the assembly according to the invention, the section of the wall of the graft vessel that is situated distally from the ring is folded over into abutment against the section of the wall of the graft vessel that is situated proximally from the ring. The prepared assembly may thus be fixed to the receiving vessel with good visibility onto the pins.

According to a still further embodiment of the invention, the device further comprises a catheter, in particular a laser catheter, wherein the graft vessel is inserted onto the distal end of the catheter. This assembly can also be prepared entirely outside the patient's body. The catheter, which has already been inserted into the graft vessel, facilitates handling of the graft vessel during the inserting of the pins into the receiving vessel.

The inventions disclosed in this application will be described hereinafter in greater detail with reference to the drawings, in which:

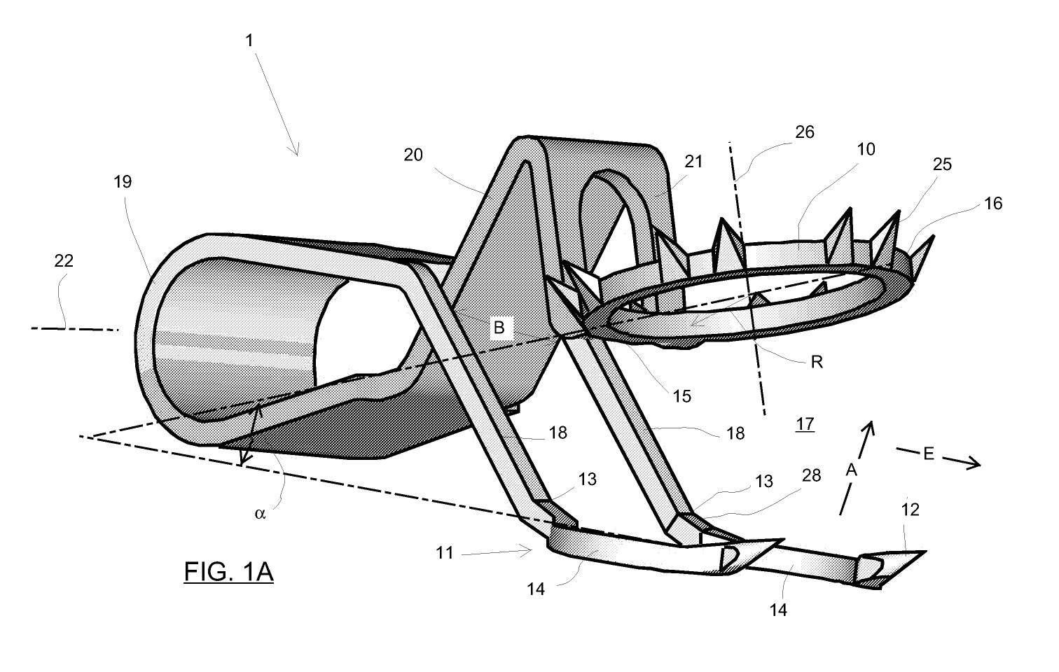

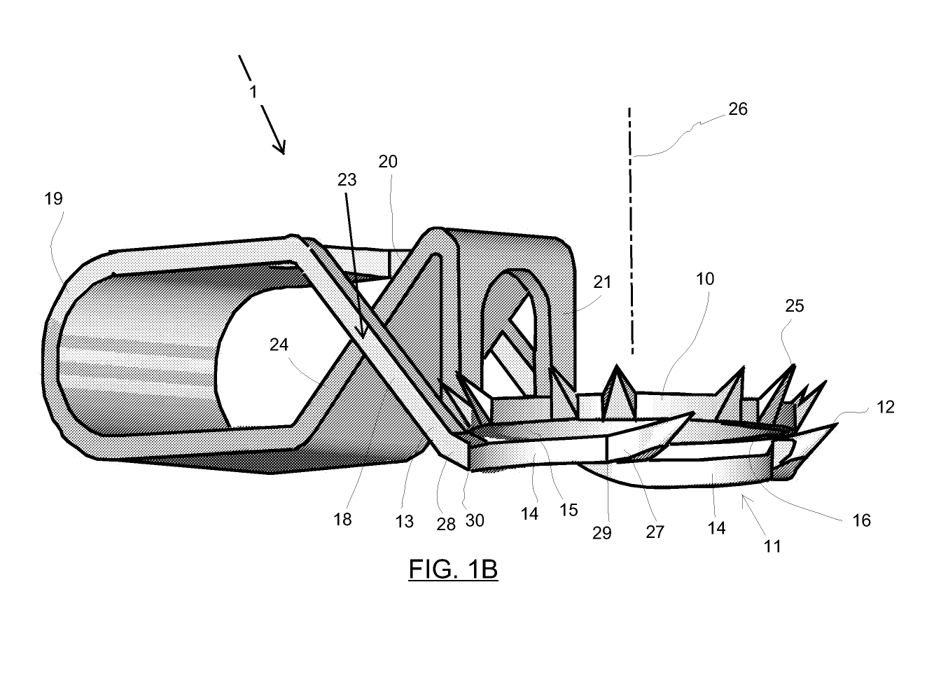

FIG. 1 is a perspective view of a first device according to the invention; FIG. 1A showing the device with the jaw opened and FIG. 1B showing the device with the jaw closed;

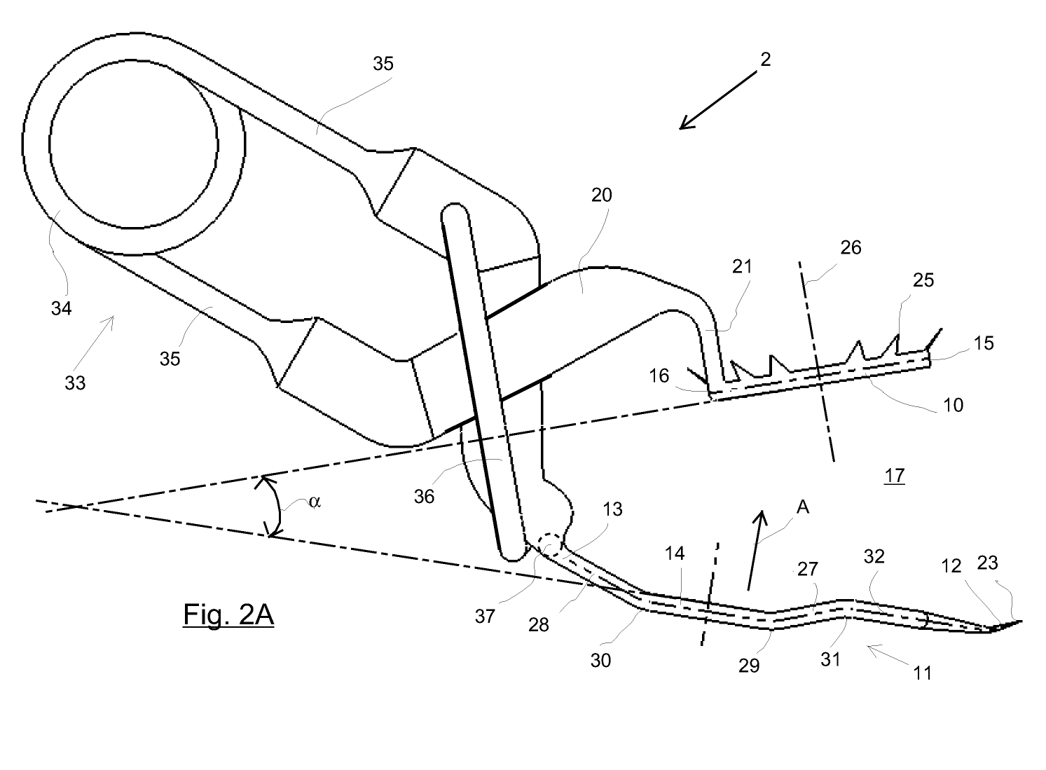

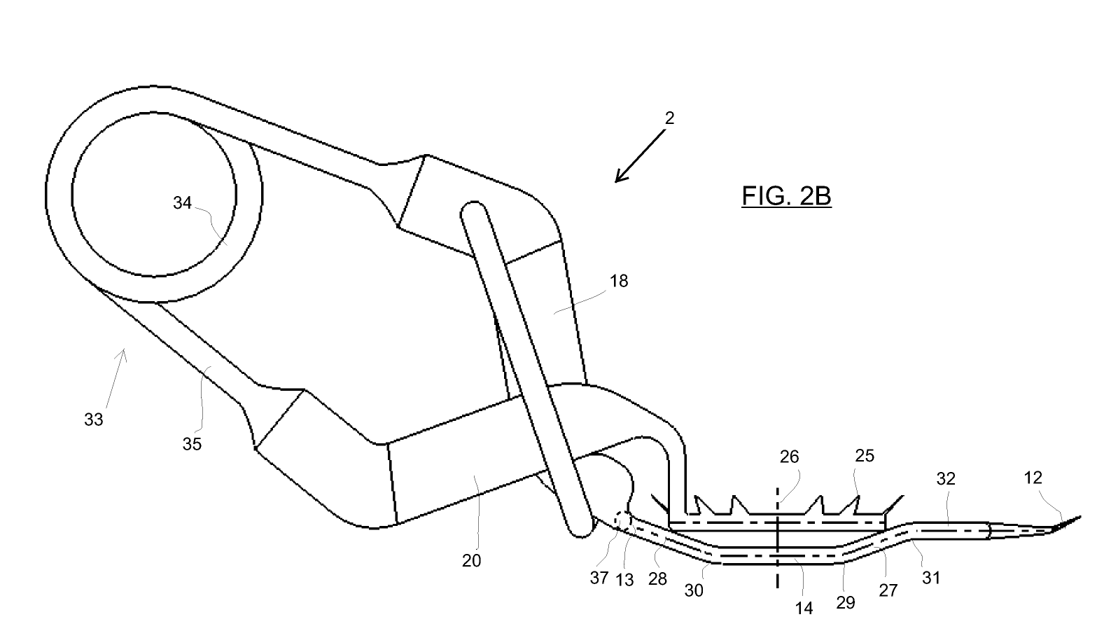

FIG. 2 is a side view of a second device according to the invention; FIG. 2A showing the device with the jaw opened and FIG. 2B showing the device with the jaw closed;

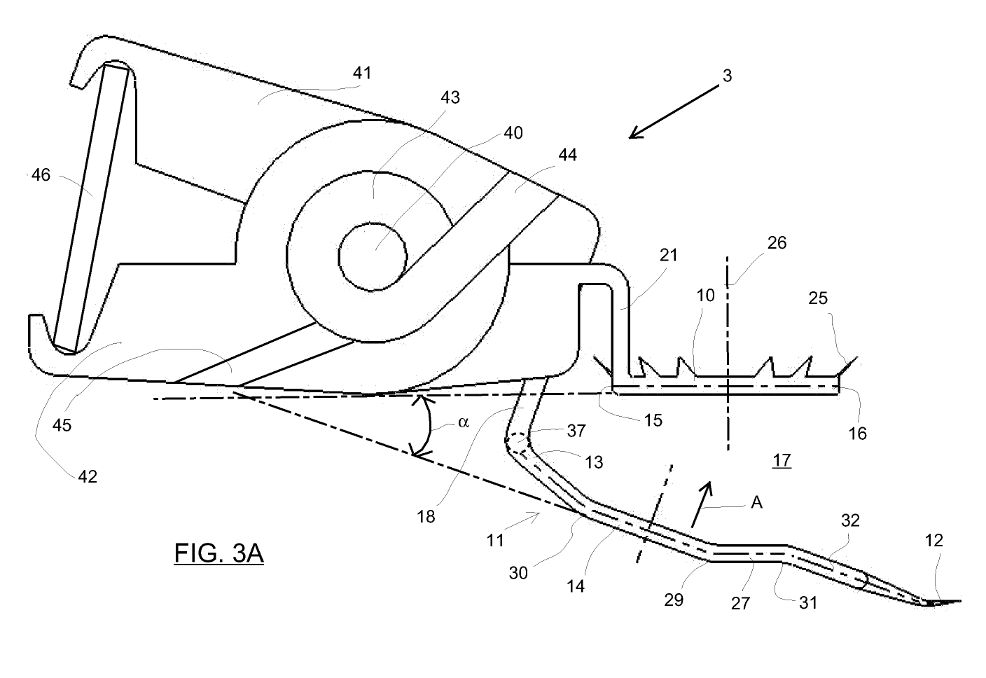

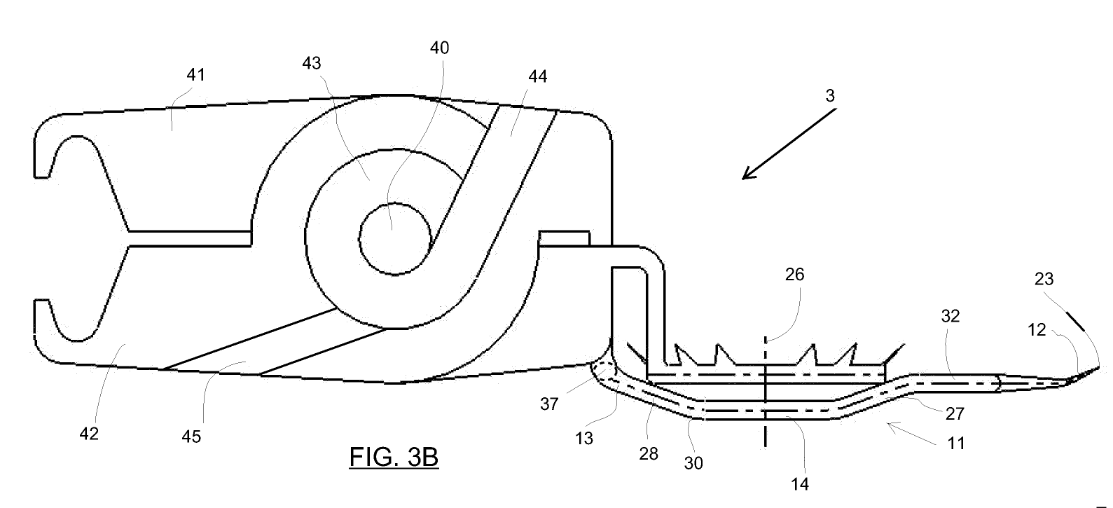

FIG. 3 is a side view of a third device according to the invention; FIG. 3A showing the device with the jaw opened and FIG. 3B showing the device with the jaw closed;

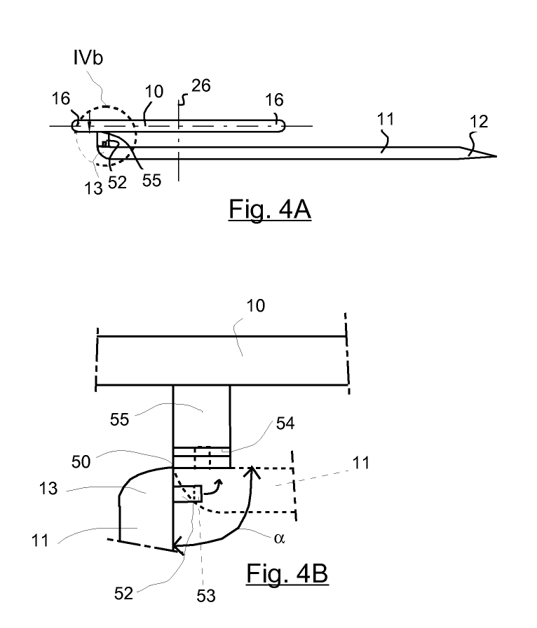

FIG. 4 is a side view of a fourth device according to the invention; FIG. 4A showing the device as a whole with the jaw closed and FIG. 4B showing the circled detail IVb from FIG. 4A with the jaw opened (solid lines) and the jaw closed (broken lines);

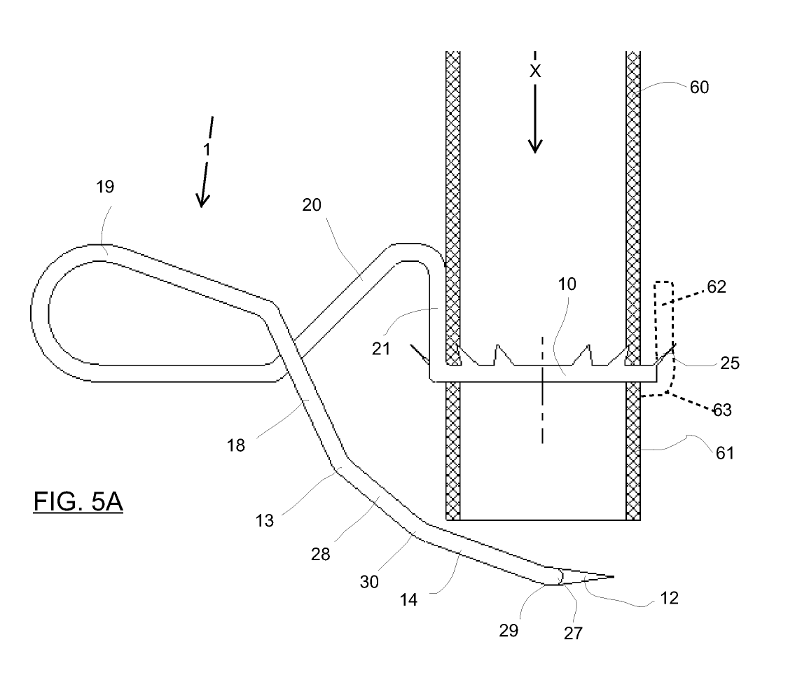

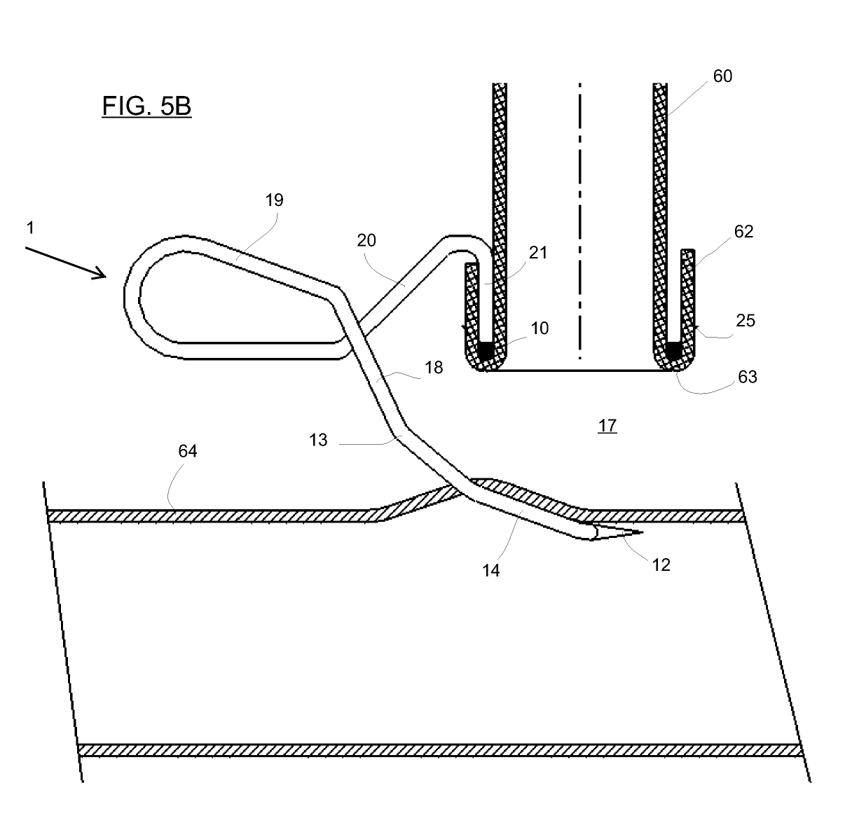

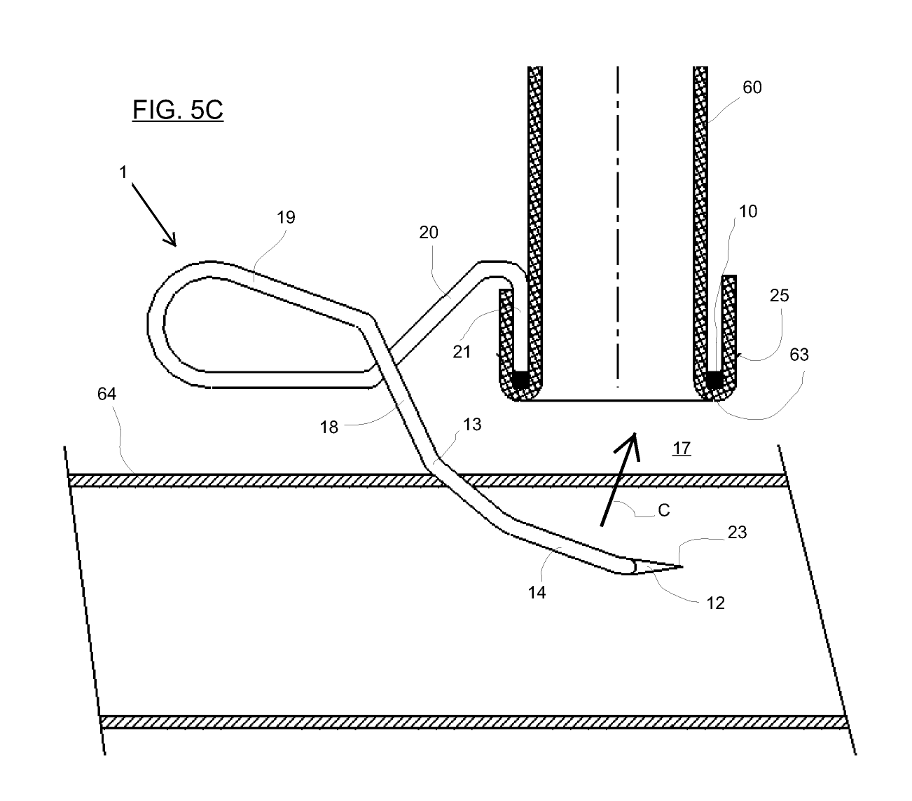

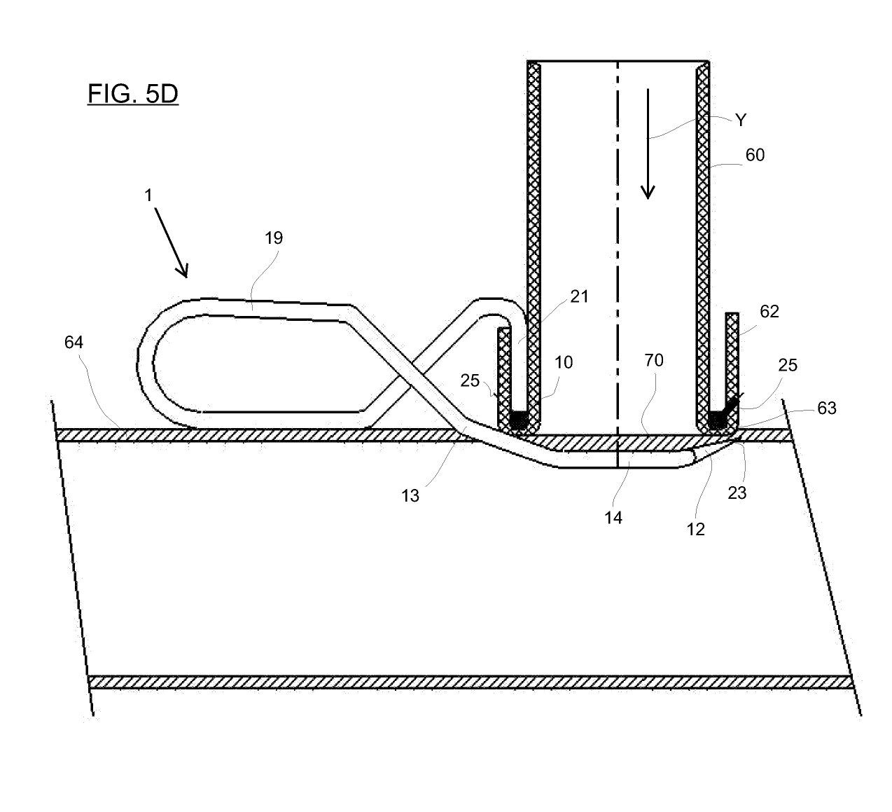

FIG. 5 shows schematically, in 4 steps (FIG. 5A, 5B, 5C and 5D respectively), how a graft vessel can be secured to a receiving vessel by means of a device according to the invention; and

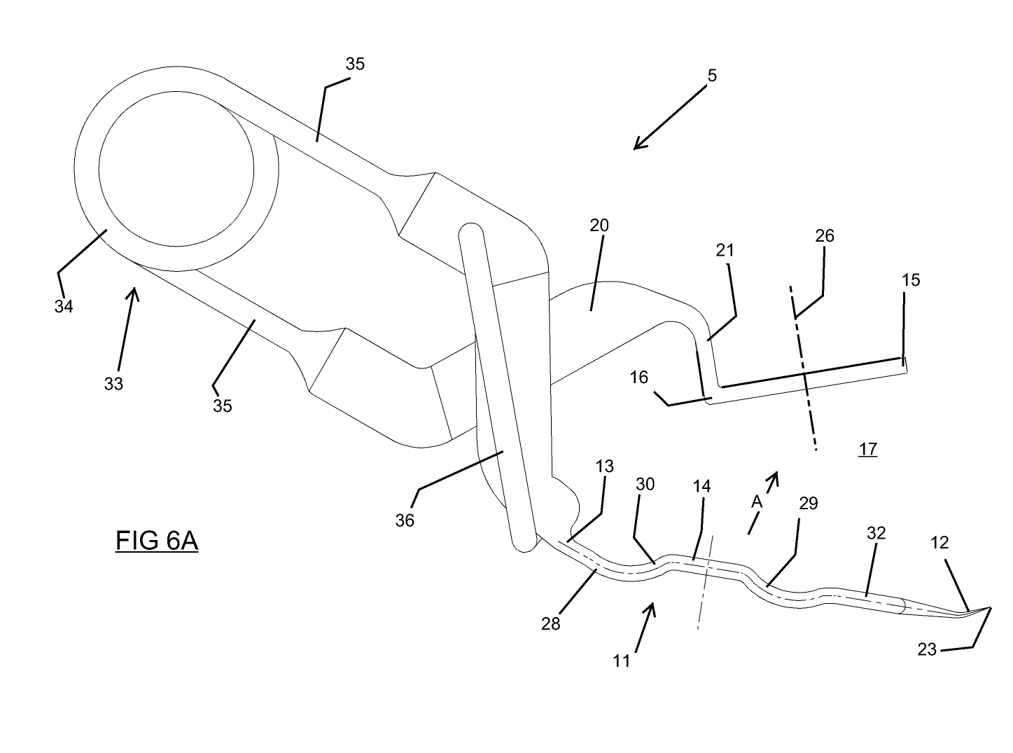

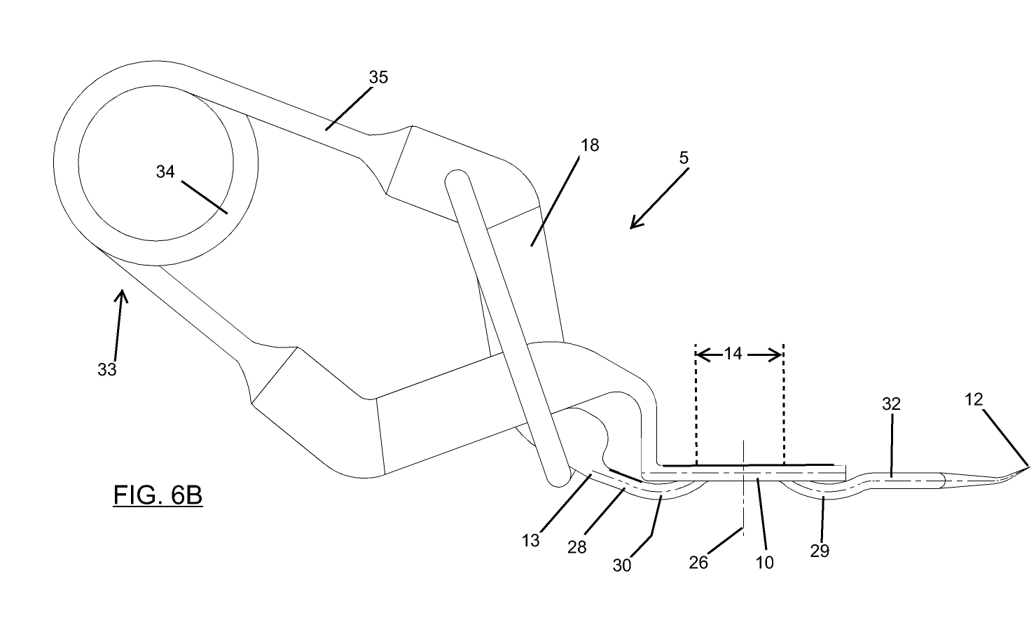

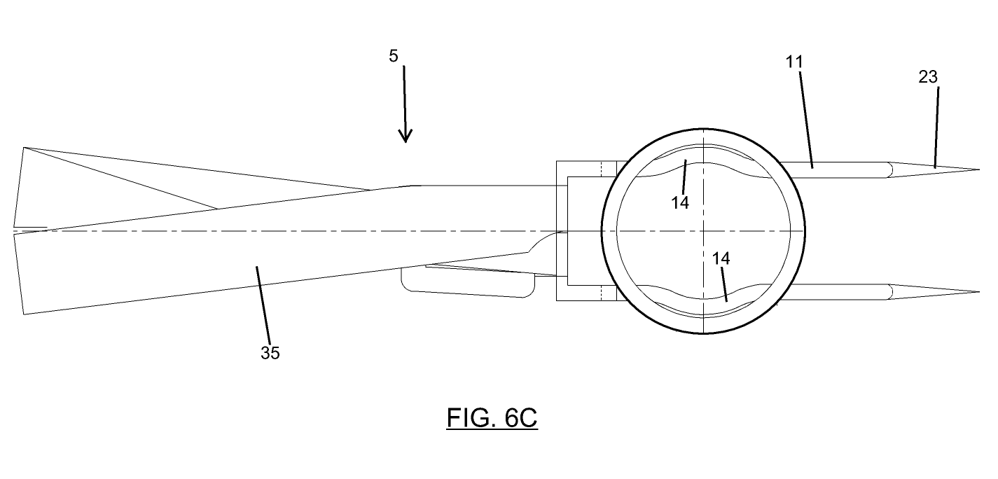

FIG. 6 shows schematically a fifth device according to the invention; FIG. 6A showing the device in side view with the jaw opened, FIG. 6B showing the device in side view with the jaw closed, and FIG. 6C showing the device in top view with the jaw closed.

FIG. 1 shows a first device according to the invention, which device is made from a single piece of sheet material which is made of a titanium alloy or of another suitable alloy or other suitable metal, is punched out and subsequently bent into the shape as shown. The device 1 comprises a ring 10 and two pins 11. Each pin has a free end 12 and a fixed end 13. The fixed end 13 of each pin 11 is secured to the ring 10 via, one after another, an oblique arm 18, a U-shaped sheet part 19, an oblique sheet part 20 and two connecting arms 21.

FIG. 1B shows the device in the first position. In the first position, the clamping sections 14 thereof run parallel to the ring—that is to say, the plane defined by the ring—and along the ring 10 from a first side 15 of the ring to a second side 16 of the ring that is situated diametrically opposite the first side 15. With reference to that which is already known from WO 2009/123434, see e.g. FIG. 8c, and also to FIG. 5d of the present invention, a vessel wall, namely a piece of the wall of the receiving vessel and a piece of the wall of the graft vessel which is turned over at the end to be fastened, will be clamped in, between the clamping sections 14 of the pins and the ring, when the device is used for fitting a graft vessel to a receiving vessel. The clamping sections 14 are here for a large section not straight but preferably run in a curved manner with a radius of curvature equal to that of the ring.

In the first position, also referred to as the closed jaw, the gap between the clamping sections 14 and the ring 10 is relatively narrow. For a sufficient clamping action, it is desirable for this gap to be narrower than the thickness of the vessel wall/vessel walls to be accommodated therebetween. In order to facilitate the introduction of vessel wall tissue into the gap, the jaw (pins 11 and ring 10) is firstly in an open position (see the second position shown in FIG. 1A), also referred to as the opened jaw.

As has also already been described in WO 2009/123434, the vessel wall of the turned-over section of the graft vessel is brought into the gap by inserting an end of the graft vessel from the side where the pins are not situated (and therefore in FIG. 1 from above) through the ring and then turning over the section of the graft vessel that protrudes below the ring. The fold formed during the turning-over is then the section of the wall of the graft vessel that enters the gap. This is easier to carry out when the jaw is opened (FIG. 1A) than when the jaw is closed (FIG. 1B). In FIG. 1B the angle α between the clamping sections 14 (more precisely, the plane defined by the clamping sections 14) and the plane defined by the ring is approximately 20°, so that the jaw is thus approximately 20° open. During the fitting of the overall entity which is thus prepared with the graft vessel to a receiving vessel—such as is shown inter alia in FIG. 8 of WO 2009/123434 and will furthermore also be described hereinafter in this application with reference to FIG. 5—the overall entity with the opened jaw (FIG. 1A) may easily be slid ‘onto the receiving vessel’ once the pins 11 have been inserted into the receiving vessel. A section of the wall of the receiving vessel then slides unimpeded into the opened jaw. In the case of a closed jaw with a narrow gap (FIG. 1B of this application and FIG. 8 of WO 2009/123434), it is quite possible that friction contact will be produced between, for example, the fold at the bottom of the graft vessel and the wall of the receiving vessel and/or between the wall of the receiving vessel and the pins. The sliding ‘onto the receiving vessel’ is hindered by this friction contact.

As will be clear from FIGS. 1A and 1B, the device 1 can easily be brought from the first position (FIG. 1B) to the second position (FIG. 1A) by squeezing together the U-shaped sheet part 19, for example between the operator's fingers, provided, that is, that the overall entity is not too small for this purpose, or using a pair of pliers. However, it is also conceivable for the device 1 to be delivered ex-works in the second position shown in FIG. 1A. A clamp can for example be placed on the U-shaped sheet part 19 for this purpose.

In order, on the one hand, to facilitate the closing of the jaw and, on the other hand, to ensure sufficient clamping when the jaw is closed, the U-shaped sheet section is embodied as a tensioning member. The spring force which is generated in the U-shaped sheet part during the compressing thereof pre-tensions the pins 11 in a pre-tensioning direction A directed toward the first position; that is to say, the pins are pre-tensioned in a direction A in order to seek to swivel toward the ring 10. The imaginary swivel axis 22 of this swivelling movement will in this case not have a fixed position but move during the swivelling. As indicated by axis 22, this swivel axis will be able to lie somewhere outside the U-shaped sheet part.

Because the points 23 of the free ends 12 of the pins 11 are situated closer together than the clamping sections 14, the pins 11 will be squeezed together somewhat during the inward insertion into the graft vessel. By making sure that the oblique sheet part has a sufficient width B at the site of, in each case, 23, where the oblique sheet part 20 intersects in the first position with the oblique arms 18, it is possible to ensure that the pins are driven apart again during the closing of the jaw. The side edges 24 of the oblique sheet part 20 then form guides for the oblique arms 18. The width B of the oblique sheet part 20 can in this case be constant over the entire length of the oblique sheet part, for example roughly equal to the distance between the points 23 in the second position, or can gradually increase in the upward direction toward zone 23. A part of the re-spreading of the pins during the closing of the jaw is also caused as a result of the fact that the distance from, on the one hand, the point of contact between the oblique arms 18 and side edges 24 of the oblique sheet part to, on the other hand, the pins 11 becomes shorter during the closing of the jaw.

As may be seen in FIGS. 1A and 1B, the ring is furthermore also provided with various protrusions 25 standing obliquely both with respect to the axial direction (axis 26) of the ring 10 and with respect to a, in each case at the site of the protrusion, radial direction R of the ring. The protrusions thus stand obliquely outward and upward. With reference to FIG. 5B, these protrusions 25 provide an anchoring for the turned-back-over end section of the graft vessel.

With reference to FIG. 1B, it may be seen that, adjoining both ends of the clamping section 14, each pin is provided with a second bending part 29 and a first bending part 30 respectively—in this case a kink—via which an oblique section 27 or 29 respectively of the pin adjoins both sides of the clamping section. In this regard, the kink is in each case approximately 22.5° in the direction of the plane defined by the ring 10, so that the pin therefore has an obliquely upwardly kinked section 27, 28 at both ends of the clamping section 14. At the free end of the pin, this obliquely kinked section merges with a pointed end, in each case via a further similar kink of approximately 22.5°. The obliquely kinked sections 27 and 28 are situated, viewed in the first position with the jaw closed (FIG. 1B) and viewed in the axial direction of the ring 10, substantially just below the body which forms the ring 10.

FIGS. 2A and 2B show a second device 2 according to the invention in the second position (jaw opened) and first position (jaw closed) respectively. This second device 2 according to the invention differs broadly in two points from the first device 1 from FIGS. 1A and 1B. These points of difference are: a) the second device 2 has longer pins and b) the tensioning mechanism. For corresponding designations, the same reference numerals, reference letters and reference symbols are therefore used in FIGS. 2A and 2B as in FIGS. 1A and 1B.

The pins 11 are lengthened in the sense that the obliquely kinked part 27 merges at 31, via a third bending part 31 (again formed here as a kink), with a straight part 32 running parallel to the clamping section 14 (or at least the plane defined by the clamping sections 14). An upwardly kinked point 23 is provided at the free end 12. The angle α between, on the one hand, the plane defined by the ring 10 and, on the other hand, the plane defined by the clamping sections 14 is approximately 25° in FIG. 2A.

The tensioning mechanism is denoted by 33 in FIGS. 2A and 2B. The U-shaped part 19 of the first device 1 is, so to speak, replaced by two legs 35 which are joined together via a 1.5 winding (in the device 1 this was, as it were, a 0.5 winding) in order to form the tensioning mechanism 33. The winding makes a higher spring force, and therefore higher pre-tensioning, possible.

Furthermore, there is also provided in the second device 2 a guide 36 which holds the oblique arms 18 and 20 laterally against each other. In the device according to FIGS. 2A and 2B, there is just an oblique arm 18, an oblique arm 20 and a connecting arm 21. The ring is carried by the single connecting arm 21. The two pins 11 are secured to a transverse arm which in FIGS. 2A and 2B is denoted schematically by 37, runs perpendicularly to the plane of the drawing and is secured to the bottom of the oblique arm 18.

FIGS. 3A and 3B show a third device 3 according to the invention in the second position (jaw opened) and first position (jaw closed) respectively. This third device 3 according to the invention differs broadly in one point from the second device 2 from FIGS. 2A and 2B. The point of difference is: the tensioning mechanism. For corresponding designations, the same reference numerals, reference letters and reference symbols are therefore used in FIGS. 3A and 3B as in FIG. 1A, 1B, 2A and 2B.

The device 3 according to the invention is embodied as a second pair of pliers, the jaw of the pair of pliers being formed by the ring 10 and the pins 11 and the grip/operating means of the pliers consisting of two parts 41 and 42 which are hingingly connected via a hinge pin 40, and a spring 43, which acts at one end 44 on the part 41 and at the other end 45 on the part 42, being wound around the hinge pin 40. In FIG. 3A the angle α is approximately 30°. FIG. 3A shows furthermore a release member 46 which is placed between the free ends of the grip/operating means and absorbs the pre-tensioning A which acts in the second position for the closing of the jaw, in order to prevent the closing. When the release member 46 is removed, the pre-tensioning of the spring 43 presses the jaw shut.

FIG. 4 shows a fourth embodiment of the invention, which is derived from FIG. 5, in particular FIG. 5B, of WO 2009/123434. For more complete representation and description, reference is therefore also made to WO 2009/123434 which is incorporated into the present application by reference.

The pin 11 is secured, in the device 4 at location 50, hingingly to the stump 55 and can thus swivel open to 90°—as shown in FIG. 4B—or even more. The angle α is thus 90° here. There is no tensioning mechanism, so that pins 11 cannot pass automatically from the second position to the first position. However, the pins can be brought by hand or by means of an instrument or other auxiliary means to the position shown in FIG. 4A. With reference to FIG. 8C of WO 2009/123434, it will be clear that the pins 11, once they have been inserted into the receiving vessel in the position shown there, can be fixed by pulling-through, for example at reference 31 in FIG. 8 of WO 2009/123434, a stitching wire below the pin and by tying it together in a knot outside the receiving vessel or by using a clip at that location or in a different manner. A holding mechanism to prevent return of the pins from the first position to the second position thus does not have to be integrated into the device according to the invention. This can be a separately positionable component. For practical reasons, the holding mechanism will generally be fitted beforehand to the remainder of the device according to the invention (that is to say, be integrated therein). As depicted in FIG. 4, the holding mechanism can be a simple lock consisting of a lip 52 which is fitted to the pin and has a ridge 53 (which is situated in FIG. 4 at the back out of sight behind the lip and is indicated in FIG. 4B by means of a dashed line) which can engage in an interlocking manner with a groove 54 which is formed on the stump 55.

It will be clear from the foregoing that, when the jaw is opened, the angle α is at least 5°, preferably at least 10°, but that the angle will from a practical perspective be at most 180°. Provision is made for the angle α to be in practice in the range of from 5° to 45° inclusive, such as in the range of from 10° to 45° inclusive or in the range of from 10° to 30° inclusive.

With reference to FIGS. 5A, 5B, 5C and 5D, it will be explained hereinafter how firstly a graft vessel can be secured to the ring and how subsequently this overall entity can be secured to the receiving vessel. This is illustrated based on the device 1 as shown in FIGS. 1A and 1B.

FIG. 5A shows the device 1 in the second position with the jaw opened. In this position, as indicated by arrow X, the distal end 61 of the graft vessel 60 is inserted through the ring 10. Subsequently, the part of the graft vessel 60 that protrudes below the ring is turned over and turned back over approximately 180°, as indicated by dashed lines on the right-hand side of FIG. 5A. The protrusions 55 pierce in this case through the turned-back part 62 of the graft vessel 60 and thus hold the turned-back part 62 fixed.

With reference to FIG. 5B, the pins 11 are subsequently inserted through the wall of the receiving vessel 64 in order afterwards to be pushed-through into the position shown in FIG. 5C. The pre-tensioning force which has built up in the U-shaped part 19 can then be released, after which the pins 11 will swivel upward, as indicated by arrow C, toward the ring 10, while in the meantime the ring, with the graft vessel fitted, is drawn toward the receiving vessel. With reference to FIG. 5D, the point 23 will become fixed in the inner side of the wall of the receiving vessel 64 and thus ensure secure fixing. The graft vessel 60 is pressed with its underside, formed by the fold 63 which has been produced during the turning-over, in a robust manner—as a consequence of a pre-tensioning which still remains in position 1—against the outer side of the receiving vessel. This pressing-on is uniform everywhere, thus ensuring a good seal all the way round. If desired, the seal can be improved still further by applying a suitable tissue glue as a sealant along the connection of the graft vessel to the receiving vessel. In fact, the seal need not be permanent because the graft vessel will grow together with the receiving vessel over the course of time. In accordance with the ELANA technique, a laser catheter can then be inserted, as indicated by arrow Y in FIG. 5D—see also WO 2009/123434 to which reference has frequently been made hereinbefore—into the graft vessel in order to burn away the tissue disc 70 in the extension of the graft vessel out of the wall of the receiving vessel 64 for producing a through-flow connection between the graft vessel and receiving vessel. If desired, this tissue disc 70 can also be removed in a different manner.

FIGS. 6A, 6B and 6C show a fifth device 5 according to the invention in the second position (jaw opened, FIG. 6A) and first position (jaw closed, FIGS. 6B and 6C). This fifth device 5 according to the invention differs broadly in two points from the second device 2 from FIGS. 2A and 2B. These points of difference are: a) the protrusions 25 are absent (note however, that optionally these can also be present) and b) the clamping sections 14 of the pins 11 are configured differently. For corresponding designations, the same reference numerals, reference letters and reference symbols are therefore used in FIGS. 1-5.

Each pin 11 is provided, between the clamping section 14 of the pin 11 and the fixed end 13, with a first bending part 30. This first bending part 30 differs from the first bending part 30 of the other embodiments. The first bending part 30 of this fifth embodiment bends, viewed in the first position and viewed from the clamping section 14 toward the fixed end 13, from within the space defined by the ring 10 downwardly to pass underneath the ring 10. Thus in first position, the clamping section 14 lies within the ring 10. This improves the clamping action between the ring and clamping sections. Further, when using the ELANA technique, the clamping section lying inside the ring can serve as abutment for the lower end of the laser catheter to prevent the laser catheter from being inserted into the target vessel and/or as stop for the laser beams to prevent them from entering the target vessel. Finally, this clamping section allows application of the device on smaller target vessels. In order to allow the pins 11 also to extend on the otherside outside the ring, each pin 11 is provided, between the clamping section 14 of the pin 11 and the free end 12, with a second bending part 29.

This second bending part 29 differs from the second bending part 29 of the other embodiments. Viewed in the first position and viewed from the clamping section 14 toward the free end 12, the second bend 29 of the fifth embodiment bends from within the space defined by the ring 10 downwardly to pass underneath the ring 10. Outside the ring 10, the second bending part 29 bends upward again like the second bending part of the other embodiments. It is noted that in the fifth embodiment the clamping section 14 is the part of the in extending about straight and lying within the ring (when in first position). As such this clamping section 14 is a little shorter than in the other embodiments, however it is to be noted that also the first bending part 30 and second bending part 29 of the fifth embodiment provide clamping.

With respect to the fifth embodiment it is further noted that the first bending part 30 and second bending part 29 might be left away so that, when in first position, the clamping section lies below the ring 10 instead of inside the ring 10. Taking into account that the clamping parts 14 curve along a radius smaller than the radius of the ring and that the clamping parts 14 extend in first position still parallel to and along the ring 10, this means that the clamping parts in this situation still might serve for an abutment for a laser cathether and/or a stop for the laser beams. Further also this modified fifth embodiment allows use on smaller target vessels.

The inventions disclosed in this application may also be expressed as outlined in the following clauses:

1] Device (1, 2, 3, 4) for producing an ETS anastomosis connection between an end of a graft vessel (60) and the side wall of a receiving vessel (64) and/or a STS anastomosis connection between a graft vessel and the side wall of a receiving vessel, the device comprising a ring (10) and two pins (11);
each pin (11) having a free end (12) and a fixed end (13) which is secured to the ring (10); the pins (11) extending, viewed from the fixed end (13) toward the free end (12), next to and set apart from each other in the same direction;
the pins (11) each having a clamping section (14) where the pin (11) extends, in a first position, parallel to and along said ring (10) from a first side (15) of the ring (10) to a second side (16) of the ring (10) that is situated diametrically opposite said first side (15) in order to be able to clamp tissue of the wall of the receiving vessel (64) between the ring (10) and the clamping sections (14);
characterized
in that, in a second position, the distance from the clamping sections of the pins (11) to the ring (10) is greater than in the first position in order to form, between the pins (11) and the ring, an opened jaw (17), and
in that the pins (11) are movable, from the second position to the first position, toward the ring (10) in order to close the jaw.
2] Device (1, 2, 3, 4) according to clause 1, wherein the clamping sections (14) of the pins (11) are, in a second position, at an angle (α) with respect to the plane defined by the ring (10) in order to form, together with the ring (10), an aforementioned jaw (17) which widens, viewed in the direction from the fixed ends (13) toward the free ends (12); and wherein the pins (11) are swivellable, with respect to the ring (10), by means of a swivelling movement from the second position to the first position for closing the jaw (17).
3] Device (1, 2, 3, 4) for producing an ETS anastomosis connection between an end of a graft vessel (60) and the side wall of a receiving vessel (64) and/or a STS anastomosis connection between a graft vessel and the side wall of a receiving vessel, the device comprising a ring (10) with two pins (11);
each pin (11) having a free end (12) and a fixed end (13) which is secured to the ring (10);
the pins (11) extending, viewed from the fixed end (13) toward the free end (12), next to and set apart from each other in the same direction;
the pins (11) each having a clamping section where the pin (11) extends, in a first position, parallel to and along said ring (10) from a first side (15) of the ring (10) to a second side (16) of the ring (10) that is situated diametrically opposite said first side (15) in order to be able to clamp tissue of the wall of the receiving vessel (64) between the ring (10) and the clamping sections (14);
characterized
in that the clamping sections (14) of the pins (11) are, in a second position, at an angle (α) with respect to the plane defined by the ring (10) in order to form, together with the ring (10), a jaw (17) which widens, viewed in the direction from the fixed ends (13) toward the free ends (12);
in that the pins (11) are swivellable, with respect to the ring (10), by means of a swivelling movement from the second position to the first position for closing the jaw (17).
4] Device (1, 2, 3, 4) according to clause 1 or 2, wherein, in the second position, the angle (α) of the clamping sections (14) with respect to the ring (10) is at least 5°, such as at least 10°.
5] Device (1, 2, 3, 4) according to one of the preceding clauses 2-4, wherein, viewed parallel to the plane defined by the ring (10), the distance from the swivel axis (22) of the aforementioned swivelling movement to a transverse axis, running parallel to the swivel axis (22), through the centre of the ring (10) is at least 70%, more particularly at least 90%, of the radius (R) of the ring (10).
6] Device (1, 2, 3, 4) according to one of the preceding clauses, wherein, viewed parallel to the plane defined by the ring (10), the distance from the swivel axis (22) of the aforementioned swivelling movement to a transverse axis, running parallel to the swivel axis (22), through the centre of the ring (10) is greater than the radius (R) of the ring (10), more particularly is greater than 2×the radius (R) of the ring (10).
7] Device (1, 2, 3, 4) according to one of the preceding clauses, further comprising a holding mechanism (19; 33; 43; 53, 54) configured to counteract return of the pins (11) from the first position to the second position.
8] Device (1, 2, 3, 4) according to clause 7, wherein the holding mechanism comprises a lock (53, 54), such as a snap or ratchet mechanism with one or more locking positions.
9] Device (1, 2, 3, 4) according to one of clauses 7 or 8, wherein the holding mechanism comprises a tensioning member (19, 33, 43) which pre-tensions the pins (11) from the second position in a pre-tensioning direction (A) directed toward the first position.
10] Device (1, 2, 3, 4) according to clause 9, wherein the tensioning member (19, 33, 43) pre-tensions, in the first position, the pins (11) in the aforementioned pre-tensioning direction (A).
11] Device (1, 2, 3, 4) according to one of clauses 9-10, wherein the tensioning member (19, 33, 43) comprises a bent sheet or wire part, in particular a sheet or wire part made of metal.
12] Device (1, 2, 3, 4) according to clause 11, wherein the sheet or wire part comprises at least one arc of a circle-shaped winding (34).
13] Device (1, 2, 3, 4) according to one of clauses 9-12, further comprising a release member (46) which is operable from a blocking position to a release position, wherein the pins (11), in the blocking position, are in the second position and are prevented from swivelling from the second position to the first position and, in the release position, are released to swivel to the first position.
14] Device (1, 2, 3, 4) according to one of the preceding clauses, wherein each pin (11) is provided, between the clamping section of the pin (11) and the fixed end (13), with a first bending part (30) which, viewed from the clamping section toward the fixed end (13), bends away in the direction of the plane defined by the ring (10), in particular over an angle of at least 20°.
15] Device (1, 2, 3, 4) for producing an ETS anastomosis connection between an end of a graft vessel (60) and the side wall of a receiving vessel (64) and/or a STS anastomosis connection between a graft vessel and the side wall of a receiving vessel,
the device comprising a ring (10) and two pins (11);
each pin (11) having a free end (12) and a fixed end (13) which is secured to the ring (10);
the pins (11) extending, viewed from the fixed end (13) toward the free end (12), next to and set apart from each other in the same direction;
the pins (11) each having a clamping section where the pin (11) extends parallel to and along said ring (10) from a first side (15) of the ring (10) to a second side (16) of the ring (10) that is situated diametrically opposite said first side (15) in order to be able to clamp tissue of the wall of the receiving vessel (64) between the ring (10) and the clamping sections (14);
characterized in that each pin (11) is provided, between the clamping section of the pin (11) and the fixed end (13), with a first bending part (30) which, viewed from the clamping section toward the fixed end (13), bends away in the direction of the plane defined by the ring (10), in particular over an angle of at least 20°.
16] Device (1, 2, 3, 4, 5) according to one of the preceding clauses 1-14, wherein each pin (11) is provided, between the clamping section (14) of the pin (11) and the fixed end (13), with a first bending part (30) which, in the first position and viewed from the clamping section (14) toward the fixed end (13), bends from within the space defined by the ring (10) downwardly to pass underneath the ring (10).
17] Device (1, 2, 3, 4, 5) for producing an ETS anastomosis connection between an end of a graft vessel (60) and the side wall of a receiving vessel (64) and/or a STS anastomosis connection between a graft vessel and the side wall of a receiving vessel, the device comprising a ring (10) and two pins (11);
each pin (11) having a free end (12) and a fixed end (13) which is secured to the ring (10); the pins (11) extending, viewed from the fixed end (13) toward the free end (12), next to and set apart from each other in the same direction;
the pins (11) each having a clamping section where the pin (11) extends parallel to and along said ring (10) from a first side (15) of the ring (10) to a second side (16) of the ring (10) that is situated diametrically opposite said first side (15) in order to be able to clamp tissue of the wall of the receiving vessel (64) between the ring (10) and the clamping sections (14);
characterized in that each pin (11) is provided, between the clamping section (14)_of the pin (11) and the fixed end (13), with a first bending part (30) which, in the first position and viewed from the clamping section (14) toward the fixed end (13), bends from within the space defined by the ring (10) downwardly to pass underneath the ring (10).
18] Device (1, 2, 3, 4) according to one of clauses 14-17, wherein the first bending part (30) is located, in the first position and viewed transversely to the plane defined by the ring (10), substantially just below the ring (10).
19] Device (1, 2, 3, 4) according to one of the preceding clauses, wherein each pin (11) is provided, between the clamping section of the pin (11) and the free end (12), with a second bending part (29) which, viewed from the clamping section toward the free end (12), bends away in the direction of the plane defined by the ring (10), in particular over an angle of at least 20°.
20] Device (1, 2, 3, 4) for producing an ETS anastomosis connection between an end of a graft vessel (60) and the side wall of a receiving vessel (64) and/or a STS anastomosis connection between a graft vessel and the side wall of a receiving vessel,
the device comprising a ring (10) and two pins (11);
each pin (11) having a free end (12) and a fixed end (13) which is secured to the ring (10);
the pins (11) extending, viewed from the fixed end (13) toward the free end (12), next to and set apart from each other in the same direction;
the pins (11) each having a clamping section where the pin (11) extends parallel to and along said ring (10) from a first side (15) of the ring (10) to a second side (16) of the ring (10) that is situated diametrically opposite said first side (15) in order to be able to clamp tissue of the wall of the receiving vessel (64) between the ring (10) and the clamping sections (14);
characterized in that each pin (11) is provided, between the clamping section of the pin (11) and the free end (12), with a second bending part (29) which, viewed from the clamping section toward the free end (12), bends away in the direction of the plane defined by the ring (10), in particular over an angle of at least 20°.
21] Device (1, 2, 3, 4, 5) according to one of the preceding clauses, wherein each pin (11) is provided, between the clamping section (14) of the pin (11) and the free end (12), with a second bending part (29) which, in the first position and viewed from the clamping section (14) toward the free end (12), bends from within the space defined by the ring (10) downwardly to pass underneath the ring (10).
22] Device (1, 2, 3, 4, 5) for producing an ETS anastomosis connection between an end of a graft vessel (60) and the side wall of a receiving vessel (64) and/or a STS anastomosis connection between a graft vessel and the side wall of a receiving vessel,
the device comprising a ring (10) and two pins (11);
each pin (11) having a free end (12) and a fixed end (13) which is secured to the ring (10); the pins (11) extending, viewed from the fixed end (13) toward the free end (12), next to and set apart from each other in the same direction;
the pins (11) each having a clamping section where the pin (11) extends parallel to and along said ring (10) from a first side (15) of the ring (10) to a second side (16) of the ring (10) that is situated diametrically opposite said first side (15) in order to be able to clamp tissue of the wall of the receiving vessel (64) between the ring (10) and the clamping sections (14);
characterized in that each pin (11) is provided, between the clamping section (14) of the pin (11) and the free end (12), with a second bending part (29) which, in the first position and viewed from the clamping section (14) toward the free end (12), bends from within the space defined by the ring (10) downwardly to pass underneath the ring (10).
23] Device (1, 2, 3, 4) according to one of clauses 19-22, wherein the second bending part (29) is located, in the first position and viewed transversely to the plane defined by the ring (10), substantially just below the ring (10).
24] Device (1, 2, 3, 4) according to one of clauses 19-23, wherein the pin (11) continues, from the second bending part (29) in the direction of the plane defined by the ring (10), up to the free end (12) and wherein the free end (12) is provided with a point which, in the first position, ends in the plane defined by the ring (10) or protrudes above the plane defined by the ring (10).
25] Device (1, 2, 3, 4) according to one of clauses 19-24, wherein, viewed in the direction of the free end (12) of the pin, the second bending part is followed by a third bending part (31) which bends the pin (11) back, in the opposite direction, to a section running substantially parallel to the clamping section.
26] Device (1, 2, 3, 4) according to clause 25, wherein the section which is bent back to substantially parallel to the clamping section extends, in the first position, in the plane defined by the ring (10).
27] Device (1, 2, 3, 4) according to clause 26, wherein the free end (12) is provided with a point which protrudes, in the first position, from the section which is bent back to substantially parallel to the clamping section obliquely upward above the plane defined by the ring (10).
28] Device (1, 2, 3, 4) according to one of the preceding clauses, wherein the ring (10) is provided with a plurality of protrusions (25) which are attached distributed over the circumference, stand obliquely with respect to the axial and radial direction of the ring (10) and point in a direction away from the pins (11), which protrusions (25) are in particular pointed.
29] Device (1, 2, 3, 4) for producing an ETS anastomosis connection between an end of a graft vessel (60) and the side wall of a receiving vessel (64) and/or a STS anastomosis connection between a graft vessel and the side wall of a receiving vessel, the device comprising a ring (10), characterized in that the ring (10) is provided with a plurality of protrusions (25) which are attached distributed over the circumference and stand obliquely with respect to the axial and radial direction of the ring (10), which protrusions (25) are in particular pointed.
30] Device (1, 2, 3, 4) according to clause 25, wherein the device (1, 2, 3, 4) further comprises two pins (11); each pin (11) having a free end (12) and a fixed end (13) which is secured to the ring (10); the pins (11) extending, viewed from the fixed end (13) toward the free end (12), next to and set apart from each other in the same direction; the pins (11) each having a clamping section where the pin (11) extends parallel to and along said ring (10) from a first side (15) of the ring (10) to a second side (16) of the ring (10) that is situated diametrically opposite said first side (15) in order to be able to clamp tissue of the wall of the receiving vessel (64) between the ring (10) and the clamping sections (14).
31] Device (1, 2, 3, 4) according to clause 30, wherein the protrusions (25) point in a direction away from the pins (11).
32] Device (1, 2, 3, 4) according to clause 31, further comprising a graft vessel (60) which is inserted with a distal end through the ring (10), wherein the section of the wall of the graft vessel (60) that is situated distally from the ring (10) is folded over and is hooked onto the protrusions (25).
33] Device (1, 2, 3, 4) according to one of the preceding clauses, further comprising a graft vessel (60) which is inserted with a distal end through the ring (10).
34] Device (1, 2, 3, 4) according to clause 32 or 33, wherein the section of the wall of the graft vessel (60) that is situated distally from the ring (10) is folded over into a positioning parallel to the section of the wall of the graft vessel (60) that is situated proximally from the ring (10).
35] Device (1, 2, 3, 4) according to one of clauses 33-34, further comprising a catheter, in particular a laser catheter, wherein the graft vessel (60) is inserted onto the distal end of the catheter.

As will be clear from the foregoing clauses, the embodiments (claimed in the claims) with the first bending part, the second bending part and the protrusions provided on the ring can also be applied entirely independently of the pins which are swivellable with respect to the ring (and therefore without swivellable pins), the embodiment with protrusions provided on the ring can itself be applied entirely independently of any pin, and these 3 embodiments can in addition be applied entirely independently of one another and also in any desired combination with one another. Divisional patent applications may in the future still relate thereto. Furthermore, it will be clear from the foregoing clauses, in particular clause 1, that the jaw can also be closed in a manner other than with a swivelling movement. It is also conceivable, within the concept of the inventions outlined in this application, for the jaw to be closed by a translatory movement, the clamping sections of the pins therefore also running, in the second position, parallel to the ring (the plane defined by the ring). With respect to use for a side to side anastomosis it is noted that the ring can be attached to the side wall of a graft vessel as well and that the attachment to the side wall of the target vessel can be done in the same manner as described with respect to an end to side anastomosis.

It will be noted that the inventions described in this application are in no way limited to the embodiments with reference to which they are described. The scope of protection of a patent which is granted to this application or to an application divisional therefrom shall be defined by the claims of that patent as granted.

1. A device configured to produce an anastomosis connection between a graft vessel and a side wall of a receiving vessel, the device comprising a ring and two pins;
each pin including a free end and a fixed end which is secured to the ring;
the pins extending, when viewed from the fixed end toward the free end, next to and set apart from each other in the same direction;
the pins each including a clamping section, wherein the pin extends, in a first position, parallel to and along said ring from a first side of the ring to a second side of the ring that is situated diametrically opposite said first side in order to be able to clamp tissue of the wall of the receiving vessel between the ring and the clamping sections and, in a second position of the pins, the distance from the clamping sections of the pins to the ring is greater than in the first position, the pins in the second position forming an opened jaw, between the pins and the ring, the pins are being movable, from the second position to the first position, toward the ring to close the jaw; and
a holding mechanism configured to counteract return of the pins from the first position to the second position, wherein the holding mechanism includes a tensioning member configured to pre-tension the pins from the second position in a pre-tensioning direction directed toward the first position.

2. The device of claim 1, wherein the clamping sections of the pins are, in a second position, at an angle with respect to the plane defined by the ring to form, together with the ring, the jaw which widens, viewed in the direction from the fixed ends toward the free ends, and wherein the pins are swivelable about a swivel axis, with respect to the ring from the second position to the first position for closing the jaw.

3. The device of claim 2, wherein, in the second position, the angle of the clamping sections with respect to the ring is at least 5°.

4. The device of claim 2, wherein, viewed parallel to the plane defined by the ring, the distance from the swivel axis to a transverse axis, running parallel to the swivel axis, through the centre of the ring is at least 70% of a radius of the ring.

5. The device of claim 2, wherein, viewed parallel to the plane defined by the ring, the distance from the swivel axis to a transverse axis, running parallel to the swivel axis, through the centre of the ring is greater than a radius of the ring.

6. (canceled)

7. The device of claim 1, wherein the holding mechanism comprises a lock.

8. (canceled)

9. The device of claim 1, wherein the tensioning member pre-tensions, in the first position, the pins in the pre-tensioning direction.

10. The device of claim 1, wherein the tensioning member includes a bent sheet or wire part.

11. The device of claim 10, wherein the sheet or wire part comprises at least one arc of a circle-shaped winding.

12. The device of claim 1, further comprising a release member which is operable from a blocking position to a release position, wherein the pins, in the blocking position, are in the second position and are inhibited from swiveling from the second position to the first position and, in the release position, are released to swivel to the first position.

13. The device of claim 1, wherein each pin is provided, between the clamping section of the pin and the fixed end, with a first bending part which, viewed from the clamping section toward the fixed end, bends away in the direction of the plane defined by the ring.

14. The device of claim 1, wherein each pin is provided, between the clamping section of the pin and the fixed end, with a first bending part which, in the first position and viewed from the clamping section toward the fixed end, bends from within the space defined by the ring downwardly to pass underneath the ring.

15. The device of claim 13, wherein the first bending part is located, in the first position and viewed transversely to the plane defined by the ring, substantially just below the ring.

16. The device of claim 1, wherein each pin is provided, between the clamping section of the pin and the free end, with a second bending part which, viewed from the clamping section toward the free end, bends away in the direction of the plane defined by the ring.

17. The device of claim 1, wherein each pin is provided, between the clamping section of the pin and the free end, with a second bending part which, in the first position and viewed from the clamping section toward the free end, bends from within the space defined by the ring downwardly to pass underneath the ring.

18. The device of claim 16, wherein the second bending part is located, in the first position and viewed transversely to the plane defined by the ring, substantially just below the ring.

19. The device of claim 16, wherein the pin continues, from the second bending part in the direction of the plane defined by the ring up to the free end and wherein the free end is provided with a point which, in the first position, ends in the plane defined by the ring or protrudes above the plane defined by the ring.

20. The device of claim 16, wherein, viewed in the direction of the free end of the pin, the second bending part is followed by a third bending part configured to bend the pin back, in the opposite direction, to a section running substantially parallel to the clamping section.

21. The device of claim 20, wherein the section which is bent back to substantially parallel to the clamping section extends, in the first position, in the plane defined by the ring.

22. The device of claim 21, wherein the free end is provided with a point which protrudes, in the first position, from the section which is bent back to substantially parallel to the clamping section obliquely upward above the plane defined by the ring.

23. The device of claim 1, wherein the ring is provided with a plurality of protrusions distributed over a circumference of the ring, the protrusions standing obliquely with respect to an axial direction and a radial direction of the ring, the protrusions pointing in a direction away from the pins.

24. The device of claim 1, wherein the ring is configured to accept a distal end of a graft vessel through the ring.

25. (canceled)

26. The device of claim 24, comprising a catheter, wherein the catheter is configured to allow the graft vessel to be inserted onto a distal end of the catheter.

27. The device of claim 1, wherein the clamping sections are curved along a radius smaller than the radius of the ring.
